(19) United States
(12) Patent Application Publication
(10) Pub. No.: US 2012/0265090 A1
(43) Pub. Date: Oct. 18, 2012

(54) SYSTEM AND METHOD OF ACQUIRING UTERINE EMG SIGNALS AND WIRELESSLY TRANSMITTING THE SAME

(76) Inventors: Rainer J. Fink, College Station, TX (US); Jack N. McCrary, Houston, TX (US); Mark Burns, McKinney, TX (US); Robert Garfield, Goodyear, AZ (US)

(21) Appl. No.: 13/085,650

(22) Filed: Apr. 13, 2011

(51) Int. Cl. A61B 5/0488

(52) U.S. Cl. 600/546

(57) ABSTRACT

A system and method of acquiring and transmitting uterine EMG signals is disclosed, where a signal processing module processes incoming uterine EMG signals from a patient and wirelessly transmits a processed signal to an information relaying device. The information relaying device is then configured to download the processed signal and transmit the signal to a call center or health care facility for physician evaluation. The system is ambulatory, thus allowing the patient to record and transmit uterine EMG signals anywhere a satisfactory transmission may be made.

### FIELD

This disclosure relates in general to the field of acquiring and transmitting uterine EMG signals.

### BACKGROUND OF THE INVENTION

During late pregnancy and the labor process, there are generally two methods of acquiring and monitoring uterine activity. The first method involves the use of a tocodynamometer (hereinafter referred to as a “toco”). The toco is a non-invasive device fastened to the abdomen of the pregnant patient by means of an elastic strap and used to measure uterine contraction frequency. The typical toco consists of an external, strain-gauge instrument, or a pressure transducer designed to measure the stretch of the mother's stomach and indicate when a uterine contraction has occurred. When the skin stretches, the pressure transducer records an electrical signal whose waveform can be evaluated by the treating physician.

The toco, however, has many drawbacks. One disadvantage is that it is an indirect method of pressure reading and is therefore subject to many interfering influences which falsify the measuring result. Also, the toco does not function once the baby has descended down the uterus and into the birth canal where no pressure transducer is present to report pressure variations. Moreover, the toco is highly inaccurate and fails to function properly on obese patients since the pressure transducer requires that uterine contractions be transmitted through whatever intervening tissues there may be to the surface of the abdomen.

The second method involves the use of an intrauterine pressure catheter (hereinafter referred to as an “IUPC”). A typical IUPC consists of a thin, flexible tube with a small, tip-end pressure transducer that is physically inserted into the uterus next to the baby. The IUPC is configured to measure the actual pressure within the uterus and thereby indicate the frequency and intensity of uterine contractions. However, in order to place the IUPC, the amniotic sack must be ruptured so that the catheter can be inserted. Improper placement of the IUPC catheter can result in false readings and requires repositioning. Similarly, the catheter opening can become plugged and provide false information requiring the removal, cleaning and reinsertion of the IUPC, Inserting the catheter runs the risk of severely injuring the head of the baby, and also carries with it a significant infection risk. Thus, the IUPC is generally rarely used, and typically used only at term delivery.

Currently, both the toco and the IUPC require that the maternal patient be “tethered” to the monitoring system, which is typically located in a hospital or similar maternal care facility. Thus, uterine activity is presently monitored only on site at a hospital, where the maternal patient is rarely located. Continuous monitoring of the uterine activity, however, may provide the physician with valuable information as to when labor may commence.

What is needed, therefore, is a system that overcomes the above-noted disadvantages of the toco and IUPC. In particular, a system is needed that overcomes the inaccuracy of the toco, especially in instances with obese patients, and further overcomes the invasive and precarious nature of the IUPC. Moreover, a system is needed that allows for the monitoring of uterine activity while the maternal patient is located outside the confines of a hospital, especially in cases where a risk of premature labor is heightened.

### SUMMARY

Embodiments of the disclosure may provide a system for acquiring and transmitting uterine EMG signals from a patient. The system may include at least one pair of electrodes attached to the patient and configured to measure uterine EMG signals emitted by the patient. The system may also include an ambulatory signal processing module wearable by the patient and communicably coupled to the at least one pair of electrodes, wherein the signal processing module is configured to receive, process, and transmit the uterine EMG signals, and an information relaying device configured to wirelessly receive and download the uterine EMG signals from the signal processing module, and subsequently transmit the uterine EMG signals to a call center via a user interface for evaluation by a trained professional.

Embodiments of the disclosure may further provide a method of acquiring and transmitting uterine EMG signals from a patient. The method may include placing at least one pair of electrodes externally upon the patient's skin for detection of uterine EMG signals, activating an ambulatory signal processing module that is communicably coupled to the at least one pair of electrodes, wherein the signal processing module is wearable by the patient, and acquiring uterine EMG data through the at least one pair of electrodes and conveying the uterine EMG data to the signal processing module. The method may further include recording the uterine EMG data on a memory located in the signal processing module, processing the uterine EMG data in the signal processing module to obtain a processed digital signal, transmitting wirelessly the processed digital signal to an information relaying device, wherein the information relaying device is configured to download the processed digital signal, and transmit the processed digital signal from the information relaying device to a call center, wherein the processed digital signal is viewable on a user interface for evaluation by a trained professional.

### BRIEF DESCRIPTIONS OF THE DRAWINGS

The disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 illustrates an exemplary embodiment of the system wearable by a user, according to one or more embodiments of the present disclosure.

FIG. 2 illustrates a block diagram schematic of an exemplary signal processing module, according to one or more embodiments of the present disclosure.

FIG. 2A illustrates a block diagram schematic of another exemplary signal processing module with power management features, according to one or more embodiments of the present disclosure.

FIG. 3 illustrates a block circuit diagram of the exemplary internal circuitry disposed in the signal processing module as described in FIG. 2.

FIG. 4 illustrates a block diagram of an exemplary method of acquiring and transmitting a uterine EMG signal, according to one or more embodiments of the present disclosure.

### DETAILED DESCRIPTION OF THE SPECIFIC EMBODIMENTS

Although described with particular reference to monitoring, processing, and transmitting uterine activity, those with skill in the arts will recognize that the disclosed embodiments have relevance to a wide variety of areas in addition to those specific examples described below.

All references, including publications, patent applications, and patents, cited herein are hereby incorporated by reference to the same extent as if each reference were individually and specifically indicated to be incorporated by reference and were set forth in its entirety herein.

It is to be understood that the following disclosure describes several exemplary embodiments for implementing different features, structures, or functions of the invention. Exemplary embodiments of components, arrangements, and configurations are described below to simplify the disclosure; however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various FIGURES. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

The present disclosure may include a portable uterine activity monitoring system capable of accurate maternal uterine monitoring even outside of a maternal health care facility. Because of the small size of the embodiments disclosed herein, the systems and methods are designed to be ambulatory and may be remotely implemented. In one example, the system may be implemented anywhere a cell phone signal may be obtained. Such remote monitoring may be configured to periodically update a physician of the progress of the pregnancy, and more importantly, alert the physician in the event of progress toward a potential premature delivery.

By utilizing the frequency analysis techniques as described herein, a physician may be able to accurately predict when a maternal patient is about to enter the initial stages of labor. This may prove specifically advantageous in the event of premature labor since current practice finds difficulty in determining which maternal patients are at high risk for premature delivery. Once a premature labor pattern is detected, a physician may be able to implement drug protocols designed to delay the labor and delivery process, or administer drugs configured to advance the developmental progression of vital organs, such as the lungs, eyes, heart, etc., in anticipation of early delivery.

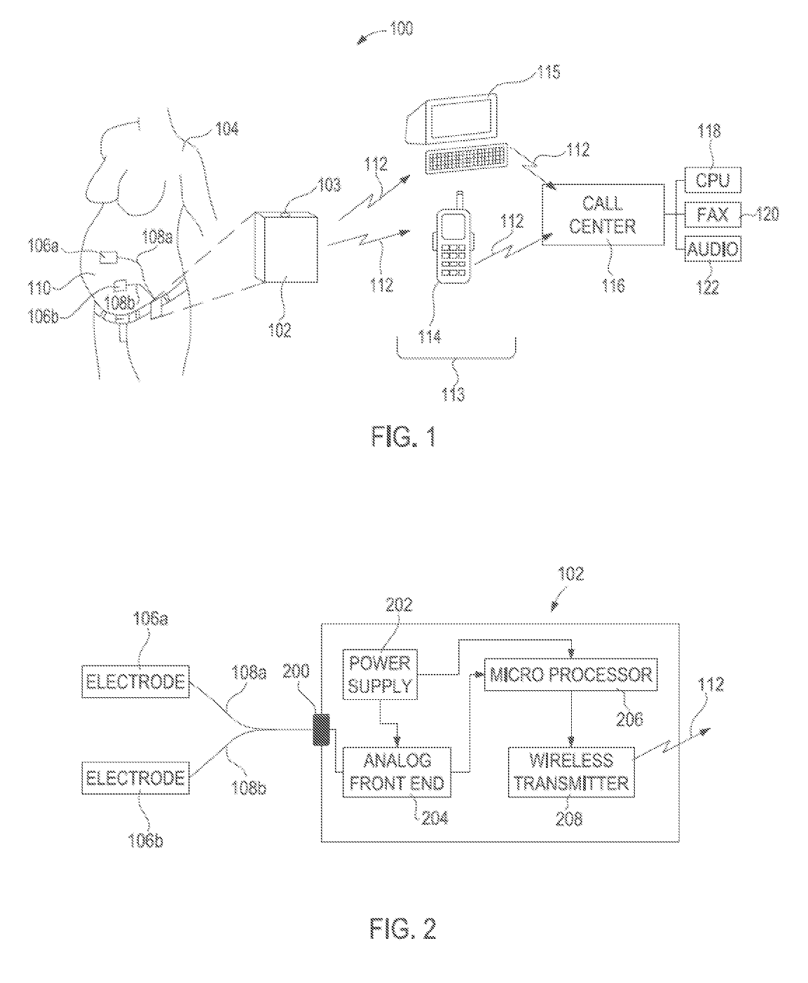

Referring now to FIG. 1, illustrated is a uterine activity monitoring system 100 for acquiring, processing, and transmitting uterine EMG signals. An EMG signal is the functional equivalent to a uterine activity signal created by a toco or IUPC, but a great deal more precise. As explanation, uterine contractions comprise coordinated contractions by individual myometrial cells of the uterus. The coordinated contraction of many myometrial cells is commonly read as an electromyography signal.

The system 100 may include a signal processing module 102 communicably coupled to a maternal patient 104 via a pair of electrodes 106a, 106b and leads 108a, 108b, respectively. In use, the electrodes 106a,b may be attached to the maternal abdomen 110 of the patient 104. As can be seen, the module 102 is dimensioned so as to be readily worn by the maternal patient 104, for example, the module 102 may be removably coupled or attached to the beltline of the maternal patient 104 by means of a clip or other fastening mechanism. Although discussed here as being a pair of electrodes, other numbers of electrodes could be utilized including odd numbers of electrodes.

In at least one embodiment, the module 102 may be activated by depressing a button 103 located on the module 102. As can be appreciated, however, activation of the module 102 may also be triggered by an external source, for example, wirelessly by the physician or at a set time interval, as explained below.

Through the electrodes 106a,b, the module 102 may be configured to process uterine electromyography (“EMG”) signals acquired from the maternal patient 104. The action potential during a uterine contraction can be measured with electrodes 106a,b placed on the maternal abdomen 110, thereby resulting in a “raw” uterine EMG signal. Specifically, the electrodes 106a,b may be configured to measure the differential muscle potential across the area between the two electrodes 106a,b and reference that potential to Vmid as described herein. Once the muscle potential is acquired, the raw uterine EMG signal, in the form of an analog wave, may then be routed through the leads 108a,b to the signal processing module 102 for processing.

In at least one embodiment, the electrodes 106a,b may be configured to employ a skin impedance compensation system (not shown). Employing a skin impedance compensation system may prove advantageous since each patient maintains a unique resistance value that can change over time. In brief, the skin impedance compensation system may be configured to measure the combined impedance of the skin of the maternal patient 104 plus the skin/electrode interface, thereby providing a relatively stable, impedance-independent output signal designed to continuously match the changing skin impedance of a maternal patient 104. Such a system is disclosed in co-pending U.S. patent application Ser. No. 12/758,552, entitled “Method and Apparatus to Determine Impedance Variations in a Skin/Electrode Interface,” the contents of which are incorporated herein by reference in their entirety.

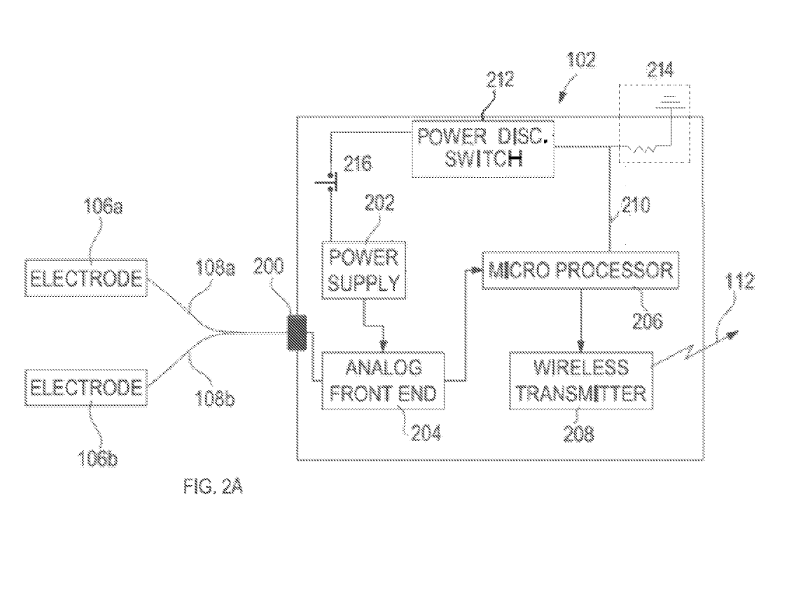

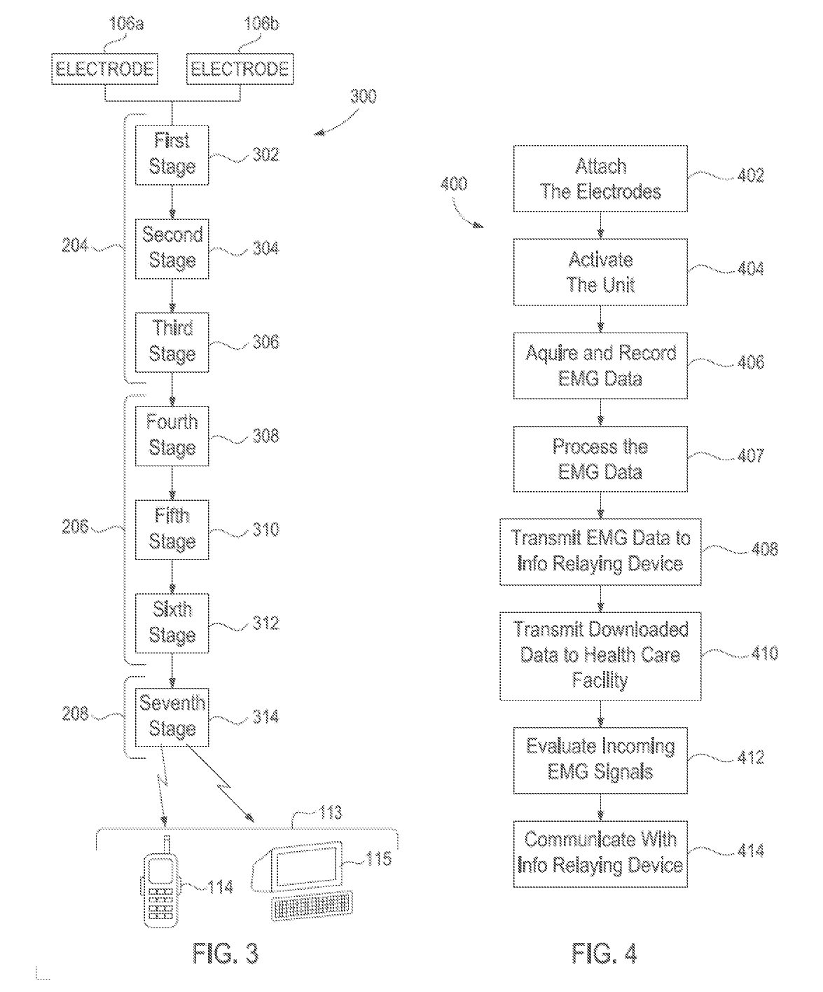

Referring now to FIG. 2, with continuing reference to FIG. 1, illustrated is block diagram of the signal processing module 102. The signal processing module 102 may house a power supply module 202, an analog front end module 204, a microprocessor 206, and a wireless transmitter 208. In exemplary operation, once a “raw” uterine EMG signal in analog format is obtained by the electrodes 106a,b, it is then channeled through an EMG communication port 200 to the analog front end 204 of the module 102. At the analog front end module 204, the incoming analog EMG signal may be preliminarily amplified and filtered, as will be described in detail below with reference to FIG. 3.

The power supply module 202 may be configured to supply power to the signal processing module 102. In an exemplary embodiment, the power supply module 202 may include a pair of AA alkaline batteries supplying, for example, +3V of potential to the module 102. In another exemplary embodiment, the power supply module 202 may also include a pair of AA nickel cadmium rechargeable batteries, a lithium ion battery, or any similarly designed battery that is capable of supplying power to the components 204, 206, 208 in the module 102. In at least one embodiment, a suitable power supply module 202 may include a +3.2V battery, rechargeable or non-rechargeable. Although specific battery voltages are listed, other voltages could also be employed.

To be able to power the components 204, 206, 208, the power supply module 202 may include a reference voltage generator circuit, as is known in the art, configured to create a reference voltage VMID. In an exemplary embodiment, the power supply module 202 may supply a voltage of +3V to the reference voltage generator circuit which may result in a reference voltage VMID of 1.5V. Throughout the signal processing module 102, each component 204, 206, 208 may be powered by and referenced to VMID.

Alternatively, since battery voltage will drop over time as the batteries run out of charge, it may be advantageous to use a voltage derived from the power supply module 202 using a voltage divider, such as VDD/2. The advantage of not using a fixed reference voltage VMID of 1.5V becomes apparent when the voltage of the batteries drops to, for example, 2.5V, at which point a fixed 1.5V VMID may be quite asymmetrical relative to the power supply 202. For instance, the amplification and filtration circuits described herein may enjoy the full 1.5V headroom on the lower side (from 0V to 1.5V), but would only have 1.0V on the positive side, thereby potentially leading to clipping the upper voltage levels of the amplified signals. To remedy this, a VDD/2 circuit with a voltage follower may be implemented so as to guarantee the same amount of overhead on the positive and negative signals.

Processed analog EMG signals from the analog front end module 204 may be conveyed to the microprocessor 206, which records the incoming signals in an internal memory (not shown). The analog uterine EMG signals may be converted into digital signals via an analog to digital (“A/D”) converter (not shown) which could be disposed in the microprocessor 206. Within the processor, the now-digital EMG signals may be digitally amplified and filtered, as will be described below. In an exemplary embodiment, the microprocessor 206 may be the commercially-available MSP430 microprocessor, but may also include any microprocessor encompassing similar characteristics.

The processed digital EMG signal may then be conveyed to the wireless transmitter 208 for transmission. As can be appreciated, other exemplary embodiments are contemplated for transmitting a variety of data options. For example, the resulting signal 112 transmitted by the wireless transmitter 208 may be the “raw” uterine EMG signal acquired from the maternal patient 104, thereby bypassing any amplification, filtration or digitization of the signal and allowing post-acquisition processing. In another embodiment, the resulting signal 112 may be the “raw” uterine EMG signal that has been converted using root-mean-square (“RMS”) calculations, thereby resulting in a unipolar EMG waveform representative of uterine activity. In another exemplary embodiment, the resulting signal 112 may include the number of contractions per minute or the amplitude of contractions recorded during a predetermined period of data acquisition.

To extend the useful battery life of the invention, it may be advantageous to employ a variety of power saving strategies. Referring now to FIG. 2A, with continuing reference to FIG. 2, illustrated is block diagram of the signal processing module 102 with additional power management features. The wireless transmitter 208 will typically use much more power than the analog front end 204 and microprocessor 206. This is because the wireless transmitter 208 must send electromagnetic waves into the air, which necessarily requires a fairly significant amount of electrical energy. To minimize the battery drain caused by the wireless transmitter 208, it may be powered down partially or fully when not actively sending data to the wireless receiver (not shown). For example, in an EMG monitor system requiring 8-bit samples to be collected and stored at a rate of only 10 samples per second, the microprocessor 206 can capture and store a large number of samples into a local memory for later transmission through the wireless transmitter 208. During the sample collection period, the wireless transmitter 208 may be powered down. In this example, it would be possible to collect and store 10 minutes of data into 6000 bytes of random access memory (RAM). Once the samples have been collected, the wireless transmitter 208 can be powered up into a data transmission mode and the block of 6000 samples can be transmitted in a matter of seconds at a high data rate. BLUETOOTH® for example, can transmit bursts of data at a rate of over 1 megabits per second. Once the block of data has been transmitted, the wireless transmitter 208 can be powered down again to conserve battery life. A power control signal that enables and disables the wireless transmitter 208 may be provided by the microprocessor 206. Such a power saving method is of most use when the delay caused by the storage and later transmission of samples is acceptable.

Although many wireless transmitter and microprocessor integrated circuits include a power saving mode to extend battery life in applications that only require a few minutes of active use per day, these lower power modes typically still require a small amount of electrical current from the battery. Even very small amounts of electrical current leaking into the signal processor 102 during long periods of inactivity may discharge the batteries in the power supply module 202 over a period of weeks or months. Therefore, it may also be advantageous for the microprocessor 206 to provide a power disconnect signal 210 to a power disconnect switch 212 which effectively disconnects the power supply module 202 from any or all active circuits in the signal processing module 102. The microprocessor might, for example, disconnect the power supply from the signal processor 102 circuits after a desired number of EMG samples have been collected and successfully transmitted. Circuits to be powered down may include the wireless transmitter 208, analog front end 204, and the microprocessor 206. The power disconnect switch 212 may take the form of an electromagnetic relay or a solid state switch. Solid state switches are readily available in the form of an integrated circuit, but might also be incorporated into the design of any of the various integrated circuits in the signal processor module 102. Since the microprocessor 206 will typically supply the power disconnect signal 210, and since this signal becomes inactive shortly after the microprocessor 206 loses its connection to the power supply, a holding resistor 214 may be employed to hold the power control signal (not shown) in the active (battery disconnected) state as soon as the microprocessor disconnects itself from the power supply 202 using the power disconnect signal 212. After the signal processor 102 is powered down in this manner, it may be powered up again by pulling the power disconnect signal 212 to its inactive (battery connected) state utilizing a user-operated momentary switch 216, for example.

Solid state switches exhibit a small amount of series resistance, which will cause noise to appear on the switched power supply signal powering the various circuit blocks of the signal processor 102. To avoid electrical noise coupling through the power supply 202 line from one circuit block of the signal processor 102 into another circuit block, it may be advantageous to employ multiple power disconnect switches (not shown), one for each critical circuit block in the signal processor 102. For example, the wireless transmitter 208 might need to be isolated from the analog front end 204 using a separate power disconnect switch (not shown). This embodiment also allows any or all power disconnect switches to be individually controlled using multiple power disconnect signals (not shown).

Referring once more to FIG. 1, through the wireless transmitter 208, the signal processing module 102 may be designed to transmit the signal 112 to a localized information relaying device 113, for example, a cellular telephone 114 or a PC 115. To accomplish this, the wireless transmitter 208 may employ a wireless application protocol (“WAP”), as is well known in the art. Although any suitable WAP may be employed, in at least one embodiment, the WAP may support BLUETOOTH® technology, and the cell phone 114, or PC may be BLUETOOTH®-enabled so as to connect the devices to communicate. Also contemplated within the disclosure is any BLUETOOTH®-enabled information relaying device 113, such as a laptop.

The information relaying device 113 may be configured to download the signal 112 and subsequently transmit the signal 112 to a call center 116. In an exemplary embodiment, the call center may include a health care facility, such as a hospital or maternal health facility. As can be appreciated, the information relaying device 113 may transmit the signal 112 via a variety of formats and locations, depending on the type of device 113 employed. For example, a cell phone 114 may utilize the standard cellular network and transmit the signals 112 wirelessly, while a PC 115 or laptop (not shown) may transmit wirelessly or wired via E-mail or other transmission medium.

To receive the resulting signal 112 from the information relaying device 113, the call center 116 may include a receiver (not shown), wireless or wired, that may be communicably coupled to at least one user interface. In an exemplary embodiment, the user interface may include a computer having a display or a monitor (“CPU”) 118, a facsimile device 120, or even speakers for receiving and projecting an audio signal 122. In at least one exemplary embodiment, the monitor 118 may be configured to display the acquired uterine EMG signals, and variations thereof, for evaluation by a trained professional, such as a physician. In another exemplary embodiment, the signals 112 may be auto-analyzed by a programmed computer designed to alert a trained professional, such as a physician, in the event incoming signals 112 breach a predetermined abnormality threshold.

Referring now to FIG. 3, with continued reference to FIG. 2, illustrated is a block diagram representative of internal circuitry 300 disposed in the signal processing module, including the analog front end module 204, the microprocessor 206, and the wireless transmitter 208. As illustrated, the internal circuitry 300 may consist of several stages configured to receive and process a raw uterine EMG signal from a maternal patient 104 (FIG. 1), and then wirelessly transmit the processed signal for analysis or display. The first three stages 302, 304, 306 may include circuitry disposed in the analog front end module 204, the subsequent three stages 308, 310, 312 may include circuitry disposed in the microprocessor 206, and the last stage 314 may include circuitry and programming disposed in the wireless transmitter 208.

The first stage 302 may include a differential to single-ended instrumentation amplifier configured to take the difference between its positive and negative inputs and reference its output to VMID. In exemplary operation, the instrumentation amplifier may be configured to take the incoming differential signal from the electrodes 106a,b and create a single-ended signal having a full-scale voltage range. To support the instrumentation amplifier in obtaining the differential amplification and creating the single-ended signal, the first stage 302 may include an arrangement of several capacitors and resistors.

The second stage 304 and the third stage 306 may include a low-pass filter and a high-pass filter, respectively. The combination of high-pass and low-pass filters may be configured to amplify and filter the incoming analog uterine EMG signals to eliminate some of the high and/or low frequency noises naturally emanating from the maternal patient 104. In an exemplary embodiment, the second stage 304 and the third stage 306 may be configured to filter the incoming analog EMG signals to frequencies located between about 0.2 Hz to about 2 Hz, the typical frequency of uterine EMG activity registered in humans. Furthermore, the filters 304, 306 may include an anti-aliasing filter combination configured to remove higher analog frequencies in preparation for digitization.

In an another exemplary embodiment, the incoming uterine EMG signals may be amplified and filtered by means of a single band-pass filter, thereby replacing the second stage 304 and the third stage 306 with a single band-pass filter stage. In this exemplary embodiment, the single band-pass filter may further perform the functions of an anti-aliasing filter, as explained above.

Within the microprocessor 206, the fourth stage 308 may include an A/D converter configured to convert the analog signal received from the analog front end 204 into a digital signal. The fifth stage 310, also included as part of the microprocessor 206, may include digital filtration and amplification processes to further clean up the converted digital signal. In particular, the fifth stage 310 may be configured to further amplify and filter the digital signal to frequencies of about 0.3 Hz to about 1 Hz. During this exemplary process, the amplification may incorporate a gain of about 1,000. The sixth stage 312 may be configured to packetize the processed data, via well-known protocol packetizing techniques, in preparation for communicating wirelessly with another device and thereby transmitting the processed signals.

It should also be noted that the microprocessor 206 may be, in at least one embodiment, time-managed by the use of a crystal oscillator. The oscillator may provide a stable clock signal for the digitally-integrated circuits mentioned above and also may stabilize the frequencies of the wireless transmitter 208.

The seventh stage 314 may include circuitry and programming disposed in the wireless transmitter 208. As explained above, the wireless transmitter 208 may employ WAP so as to communicate with an information relay device 113 (FIG. 1). The wireless transmitter 208 may utilize the commercially-available BLUECORE4® computer chip, supported by BLUETOOTH® technology. As is known in the art, the BLUECORE4® chip may be configured to convert the processed signal into an open wireless protocol, such as BLUETOOTH®, and transmit that wireless signal to the information relay device 113, for example, a cell phone 114 or PC 115. In at least one embodiment, the open wireless protocol may be capable of exchanging data over a distance of about 20 feet, and because the protocol uses a radio (broadcast) communications system, it does not have to be in line of sight of the information relay device 113.

Consistent with the BLUETOOTH® technology as a platform, the information relay device 113 may include a communication interface designed to create virtual serial ports that connect BLUETOOTH®-enabled devices. The communication interface may include, for example, a Serial Port Profile (“SPP”), which emulates serial ports in the signal processing module 102 and information relay device 113, respectively, and allows the emulated serial ports to communicate. The information relay device 113 may also be programmed to receive the signal 112 from the module 102 and subsequently transmit that signal 112 to a call center 116, as explained above. In at least one embodiment, the program may include a computer program written in JAVA® Micro Edition and subsequently downloaded to the information relay device 113 for implementation.

Moreover, operational parameters may be downloaded to the signal processing module 102 via the BLUETOOTH® SPP interface. For example, the SPP interface may be able to configure the duration of data collection (e.g., 10 min, 30 min, etc.), the packet size of the transmitted data, the transmission format (e.g., compressed data, non-compressed data, 12-bit data, 8-bit data, etc.), the sampling rate of the digitization process, and the filter coefficients (i.e., 0.3 Hz to 1 Hz) for the digital filters, as described herein.

Referring now to FIG. 4, with continued reference to FIGS. 1 and 2, an exemplary method of operation 400 for at least one embodiment of the disclosure is described. The electrodes 106a,b may be attached to the abdomen 110 of the maternal patient 104, as at 402. In at least one embodiment, data acquisition may be done outside of a hospital or maternal health care facility. The module 102 may be activated for data acquisition, as at 404. In an exemplary embodiment, the unit may be activated by manually depressing a button 103 on the module 102, but may also be activated remotely by an information relay device 113, such as a cell phone 114 or PC 115, that is programmed to communicate with the module 102 using BLUETOOTH® technology to initiate operation.

Because of the relatively small size of the module 102, the embodiments disclosed herein may be configured as ambulatory and allow for the acquisition and monitoring of uterine EMG signals at remote locations where transmission of downloaded data can be achieved. Therefore, the embodiments need not be implemented at a hospital or maternal health care facility, but may be activated directly by the maternal patient 104, for example, at home or while traveling.

Once activated, the module 102 may be configured to acquire and record a predetermined amount of uterine EMG data through the electrodes 106a,b, as at 406. In an exemplary embodiment, the module 102 may be programmed to obtain uterine EMG data for a predetermined amount of time, for example, once a day for a period of thirty minutes. As can be appreciated, however, physicians may be able to alter the program data acquisition times to suit a particular application or simply to acquire a predetermined amount of data that is not necessarily time-bound. Once the predetermined amount of data is downloaded or a predetermined amount of time for data acquisition is reached, the module 102 may be programmed to auto-terminate the data capture process, or shut down, thereby saving on battery power.

Once the uterine EMG data is acquired and recorded, the module 102 may access the internal memory and process the uterine EMG data, as at 407. In one particular embodiment, the module 102 may be configured to amplify and filter the incoming analog EMG signal to frequencies between 0.2 Hz to about 2.0 Hz. In such an embodiment, the bandwidth may be greater than the final bandwidth, with the upper cutoff remaining below about 50 Hz. Moreover, the module 102 may employ an A/D converter to convert the analog signal into a digital signal, and thereafter further amplify and filter the digital EMG signal to frequencies between 0.3 Hz to about 1.0 Hz.

After processing the uterine EMG data, the processed data may then be transmitted via BLUETOOTH® technology to an information relaying device 113 for downloading, as at 408. In an exemplary embodiment, the information relaying device 113 may include a localized cell phone 114 or a PC 115. The information relaying device 113 may then be configured to automatically transmit the downloaded uterine EMG data to a call center 116, as at 410. As can be appreciated, the information relaying device may transmit the downloaded data wirelessly or via wired communication channels.

Once the downloaded data is received at the call center 116, a physician, or other trained professional, may evaluate the incoming uterine EMG signals, as at 412. As explained above, a computer may be programmed to analyze the incoming data also, and search for abnormalities in the signal that need to be brought to the physician's attention. For example, a computer may analyze processed or unprocessed uterine EMG signals for frequencies indicating an onset of labor. Such a conclusion would then be reported to the physician to take appropriate action.

In an exemplary embodiment, in response to the incoming EMG signals, the call center 116 may be able to initiate communication with the information relaying device 113 and provide instructions either in audio or text format, as at 414. For example, if the data failed to download correctly to the call center 116, the maternal patient 104 may be alerted and instructed on how to remedy the situation. Moreover, the call center 116 may initiate communication with the information relaying device 113 to remind the maternal patient 104 of a scheduled data acquisition, or to alert the maternal patient 104 of a spike in the signals indicating the onset of labor, and then provide appropriate instructions.

In one or more embodiments, the signal processing module 102 may be configured to perform self-testing procedures, resulting in a current status indication of the module 102. The status of the module 102 may be transmitted to the receiver via the WAP interface, as described above, so that defective modules 102 can be more easily identified and replaced. Furthermore, the battery voltage may be continuously or intermittently monitored and reported throughout the collection period to ensure that the batteries do not go partially dead halfway through the data collection period. As can be appreciated, before the start of a data collection period, the batteries may have to be replaced or charged to an adequate level to assure accurate data collection. In at least one embodiment, the current voltage of the battery can be transmitted in tandem with the EMG data. As such, the signal processing module 102 may be configured to detect and flag a low battery condition so that erroneous data are not transmitted. A low-voltage message may be sent to the receiver warning of this undervoltage condition so the user 104 or physician can swap units or change/recharge the batteries.

Also contemplated within the present disclosure is a much smaller version of the signal processing module 102. With today's advances in semiconductor technology, it is clearly within the scope of the disclosure to include a signal processing module 102 that is small enough to be incorporated directly into the electrode 106a,b structure. Indeed, a miniaturized version of the module 102 may nonetheless contain the same capabilities as the module 102 described above. As can be appreciated, a much smaller power supply 202 may also be implemented, such as a lithium ion battery like those found in wristwatches. Similarly, all the microprocessing and wireless protocol may be included in a much smaller version to be able to directly transmit the processed digital signal to a nearby cellular phone 114 or equivalent device.

Moreover, the present disclosure also contemplates other exemplary embodiments where the electrodes 106a,b, or alternatively the leads 108a,b, are directly coupled to the information relaying device 113, thereby eliminating the need for a wireless transmitter 208. However, in this exemplary embodiment, the information relaying device 113 would process the incoming “raw” EMG signal. Such processing, as described above, may include the amplification and filtration of the incoming signal, and subsequently converting the analog signal into a digital signal for cellular transmission to a call center 116. In other embodiments the analog signal could be transmitted without being converted to a digital signal.

As can be appreciated, one of the many advantages of the embodiments described herein is that the maternal patient 104 is not required to be tethered to a monitoring system located in a hospital or maternal health facility, but is allowed to be located anywhere that an adequate transmission can be achieved. In an exemplary embodiment, the module 102 and information relaying device 113, as described herein, may be given to maternal patients 104 at the beginning of the second trimester. The power supply 112, as described herein, may be designed to last at least six months while in the monitoring mode.

The detailed description set forth above in connection with the appended drawings is intended as a description of exemplary embodiments in which the presently disclosed apparatus and system can be practiced. The term “exemplary” used throughout this description means “serving as an example, instance, or illustration,” and should not necessarily be construed as preferred or advantageous over other embodiments.

Further, although exemplary devices and schematics implement the elements of the disclosed subject matter have been provided, one skilled in the art, using this disclosure, could develop additional hardware and/or software to practice the disclosed subject matter and each is intended to be included herein.

In addition to the above described embodiments, those skilled in the art will appreciate that this disclosure has application in a variety of arts and situations and this disclosure is intended to include the same. As a way of example and not of limitation, other communications mediums and methods could be employed beyond BLUETOOTH®. Additionally, although certain programming languages and/or interfaces are suggested herein, other languages could be employed to reach the same ends and remain well within the scope of the preceding disclosure.

What is claimed is:

1. A system for acquiring and transmitting uterine EMG signals from a patient, comprising:
at least one pair of electrodes attached to the patient and configured to measure uterine EMG signals emitted by the patient;
an ambulatory signal processing module wearable by the patient and communicably coupled to the at least one pair of electrodes, wherein said ambulatory signal processing module is configured to receive, process, and transmit said uterine EMG signals; and
an information relaying device configured to wirelessly receive and download said uterine EMG signals from said signal processing module, and subsequently transmit said uterine EMG signals to a call center for evaluation by a trained professional via a user interface.

2. The system of claim 1, wherein said ambulatory signal processing module is additionally configured to process said uterine EMG signals after receipt but before transmission.

3. The system of claim 2, wherein said signal processing module comprises:
a power supply module;
an analog front end module;
a microprocessor; and
a wireless transmitter.

4. The system of claim 3, wherein said power supply module includes at least one battery generating a VMID voltage.

5. The system of claim 4, wherein said power supply module includes a voltage divider VDD/2 having a voltage follower, whereby a voltage output from said power supply module is uniformly regulated as at least one battery runs out of charge.

6. The system of claim 3, wherein the battery pack comprises a rechargeable battery.

7. The system of claim 3, wherein said analog front end module comprises at least one low-pass filter and at least one high-pass filter configured to amplify and filter said uterine EMG signals to a frequency located between about 0.2 Hz to about 2 Hz.

8. The system of claim 3, wherein said analog front end module comprises a band-pass filter.

9. The system of claim 3, wherein said microprocessor comprises an analog to digital converter configured to convert said uterine EMG signals from analog into a digital EMG signal.

10. The system of claim 9, wherein said microprocessor is further configured to digitally filter and amplify the digital EMG signal to a frequency of about 0.3 Hz to about 1 Hz.

11. The system of claim 1, wherein said signal processing module is activated by the patient by manually triggering a button.

12. The system of claim 1, wherein said information relaying device communicates with said signal processing module via a wireless application protocol.

13. The system of claim 12, wherein said signal processing module is remotely activated at a predetermined time by said information relaying device.

14. The system of claim 12, wherein the information relaying device comprises a BLUETOOTH-enabled cell phone, PC, or laptop.

15. The system of claim 1, wherein said user interface includes a monitor configured to display processed signals representative of uterine activity.

16. The system of claim 3, wherein said power supply module is coupled to at least one active circuit by a power disconnect switch, said power disconnect switch operable to respond to an electrical control signal to disconnect said power supply module from said at least one active circuit.

17. The system of claim 16, wherein said electrical control signal is generated by said microprocessor.

18. The system of claim 16, wherein said electrical control signal is generated by a momentary switch.

19. A method of acquiring and transmitting uterine EMG signals from a patient, comprising:
Receiving uterine EMG signals from at least one pair of electrodes;
activating an ambulatory signal processing module, said ambulatory signal processing module communicably coupled to the at least one pair of electrodes, wherein said ambulatory signal processing module is wearable by the patient;
conveying said uterine EMG signals to said signal processing module;
recording said uterine EMG signals on a memory coupled to said the signal processing module;
processing said uterine EMG signals in said signal processing module to obtain a processed digital signal;
wirelessly transmitting said processed digital signal to an information relaying device, said information relaying device configured to download said processed digital signal; and
transmitting said processed digital signal from said information relaying device to a call center, wherein said processed digital signal is made available on a user interface for review and evaluation by a trained professional.

20. The method of claim 19, wherein said signal processing module is activated remotely by the information relaying device using a wireless application protocol.

21. The method of claim 19, wherein acquiring said uterine EMG signals includes measuring uterine action potentials for a predetermined amount of time.

22. The method of claim 19, wherein processing said uterine EMG signals comprises:
amplifying and filtering said uterine EMG signals with an analog front end module to frequencies between 0.2 Hz to about 2.0 Hz, wherein said uterine EMG signals comprises an analog signal;
converting said analog signal to a digital signal with an analog to digital converter disposed in a microprocessor; and
amplifying and filtering said digital signal with said microprocessor to frequencies between 0.3 Hz to about 1.0 Hz.

23. The method of claim 19, with the additional step of initiating communication with said information relaying device by said call center, whereby the call center provides instructions to said patient.

24. The method of claim 19, wherein said information relaying device communicates with said signal processing module via a wireless application protocol.

25. The system of claim 24, wherein said signal processing module is remotely activated at a predetermined time by said information relaying device.

26. The system of claim 24, wherein said information relaying device comprises a BLUETOOTH-enabled cell phone, PC, or laptop.

27. The method of claim 19, further operable to conserve power in said ambulatory signal processing module, further comprising:
powering down a wireless transmitter;
collecting data samples during a sample collection period;
storing said data samples into said memory, said memory located in said ambulatory signal processing module, while said wireless transmitter is powered down;
powering up said wireless transmitter; and
transmitting said data samples while said wireless transmitter is powered up, whereby power supply drain in the signal processing module is minimized by only using electricity in intervals to transmit data.

28. The method of claim 19, further operable to conserve power in said ambulatory signal processing module, further comprising:
providing a power disconnect signal to a power disconnect switch; and
disconnecting a power supply module from at least one circuit in said ambulatory signal processing module, whereby electric current flow is stopped between said power supply module and any other circuitry within said signal processing module.
